(19) United States
(12) Patent Application Publication
(10) Pub. No.: US 2015/0225321 A1
(43) Pub. Date: Aug. 13, 2015

(54) Process for the manufacture of propanediol

(71) Applicant: SOLVAY SA, Brussels (BE)

(72) Inventor: Fangzheng Su, Woluwe Saint Lambert (BE)

(21) Appl. No.: 14/429,465

(22) PCT Filed: Sep. 23, 2013
(86) PCT No.: PCT/EP2013/069651

§ 371 Date: Mar. 19, 2015

(30) Foreign Application Priority Data
Sep. 26, 2012 (EP) 12186052.2

(51) Int. Cl. C07C 29/60; C08G 63/00; D01F 6/62; C08G 63/16

(52) U.S. Cl. CPC C07C 29/60; C08G 63/16; C08G 63/00; D01F 6/62; D10B 2331/04

(57) ABSTRACT

A process for manufacturing 1,3-propanediol by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form.

The present application claims benefit of European patent application n° 12186052.2 filed on Sep. 26, 2012 the content of which is incorporated herein by reference for all purposes.

Should the disclosure of any of the patents, patent applications, and publications which are incorporated herein by reference conflict with the description of the present application to the extent that it may render a term unclear, the present description shall take precedence.

The invention relates to a process for the manufacture of 1,3-propanediol, in particular for the manufacture of 1,3-propanediol by hydrogenation of glycerol.

Trimethylene glycol (1,3-propanediol) is mainly used as a building block in the production of polymers such as polytrimethylene terephthalate and it can be formulated into a variety of industrial products including composites, adhesives, laminates, coatings, moldings, aliphatic polyesters and copolyesters. It can also be used as a solvent, an antifreeze and a wood paint.

Trimethylene glycol may be chemically synthesized by the hydration of acrolein, by the hydroformylation of ethylene oxide to afford 3-hydroxypropionaldehyde, which is hydrogenated to give 1,3-propanediol, by bioprocessing of glucose and glycerol by certain micro-organisms, or by catalytic hydrogenation of glycerol.

JP 2009-275029 discloses the liquid phase hydrogenation of glycerol in the presence of a catalyst comprising iridium and an element selected from Re, Mo and W supported on various carriers. Mixtures of propanediols and propanols are obtained and a large amount of water is used as solvent. Amada et al. (Applied catalysis B: Environmental 105, 2011, 117-127) disclose that the liquid phase hydrogenation of glycerol in the presence of a Ir—ReOx/SiO2 catalyst is enhanced in the presence of sulfuric acid. The use of water and sulfuric acid complicate the overall production industrial process.

Accordingly, it is desired to provide improved processes for preparing 1,3-propanediol.

These and other problems are solved by the present invention described below.

In a first embodiment, the invention is related to a process for manufacturing 1,3-propanediol by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form.

One of the essential features of the present invention is the use of a specific zeolite as support for the iridium and rhenium compounds, with, without willing to be tied by any theory, at least one of the following advantages:

(a) To provide a supported catalyst with the suitable acidity in order to favor the selective 1,3-propanediol production;
(b) To lessen or even to avoid the use of a co-catalyst for carrying out the reaction;
(c) To provide a supported catalyst stable with respect to the iridium and rhenium compounds losses, for example by leaching or attrition;
(d) To provide a supported catalyst exhibiting a low deactivation rate;
(e) To provide a supported catalyst which can be regenerated easily;
(f) To lessen or even to avoid the use of a mineral acid, like sulfuric acid, while maintaining good yields for 1,3-propanediol, and
(g) To lessen or even to avoid the use of water without affecting negatively the 1,3-propanediol selectivity.

In a second embodiment, the invention relates to a process for making a polyester comprising obtaining 1,3-propanediol according to the process of the first embodiment of the invention and further submitting said propanediol to a reaction with a carboxylic acid and/or a carboxylic acid ester.

In a third embodiment, the invention relates to a process for making a polyester fiber comprising obtaining a polyester according to the process of the second embodiment of the invention and further converting said polyester in to a fiber.

In a fourth embodiment, the invention relates to a process for preparing a supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is partially in the hydrogen form, comprising co-impregnating said zeolite with an aqueous composition containing at least one iridium compound and at least one rhenium compound, drying said co-impregnated zeolite, calcining said dried co-impregnated zeolite under an oxygen containing gas and further treating the said calcined co-impregnated zeolite under hydrogen in water at a temperature higher than or equal to 150° C. and lower than or equal to 350° C.

In the process according to the first embodiment of the invention, the glycerol can be synthetic glycerol or natural glycerol or any mixture thereof. Synthetic glycerol is glycerol which has been obtained from non renewable raw materials. The glycerol is preferably natural glycerol i.e. glycerol which has been prepared in a conversion process of renewable raw materials. By glycerol which has been prepared in a conversion process of renewable raw materials one intends to denote glycerol obtained in a process selected from the group consisting of hydrolysis, saponification, transesterification, aminolysis and hydrogenation of oils and/or fats of animal and/or plant and/or algae origin, of fermentation, hydrogenation and hydrogenolysis of mono- and polysaccharides and derived alcohols, derived from or occurring naturally in the biomass, and any combination thereof. Glycerol which has been obtained during the manufacture of biodiesel, i.e. during the transesterification of oils and/or fats of animal and/or plant, and preferably during the transesterification of oils and/or fats of plant origin, is particularly convenient. Glycerol which has been obtained in the manufacture of biodiesel is more particularly convenient.

In the process according to the first embodiment of the invention, the hydrogen can be obtained from any source. The hydrogen is preferably molecular hydrogen.

In the process according to the first embodiment of the invention, the hydrogen is preferably obtained from at least one process selected from the group consisting of steam reforming of hydrocarbons, partial oxidation of hydrocarbons, autothermal reforming of hydrocarbons, water-gas shift, coal gasification, pyrolysis of organic waste products (tar, lignite pitch, petroleum distillation residues, plastics, rubber, cellulose, paper, textile, wood, straw, mixed municipal waste . . . ) and co-pyrolysis of organic wastes products with coal (including bituminous coal, lignite, . . . ), thermal and non-thermal plasma cracking of mixtures of water or steam, and fuels, biomass gasification, biomass pyrolysis and subsequent gasification, thermal or catalytic decomposition of nitrogen compounds like ammonia, hydrazine, biochemical hydrogen fermentation, steam reforming of alcohols, for instance monoalcohols, such methanol or ethanol, and polyols, such as propanediols and glycerine, alkaline cracking of insaturated fatty acid particularly oleic and ricinoleic acid, electrolysis of an aqueous solution of a hydrogen halide, electrolysis of an aqueous solution of a metal halide like for instance sodium chloride or potassium chloride, hydrolysis of metals or metal hydrides, water splitting from for instance alkaline electrolysis, proton-exchange membrane electrolysis, solid oxide electrolysis, high pressure electrolysis, high temperature electrolysis, photoelectrochemical water splitting, photocatalytic water splitting, photobiological water splitting and water thermolysis. When the selected process is electrolysis of an aqueous solution of a hydrogen halide, the hydrogen halide is often selected from hydrogen chloride, hydrogen fluoride and any mixture thereof, and is frequently hydrogen chloride. When the selected process is electrolysis of an aqueous solution of sodium chloride or potassium chloride, electrolysis can be any of mercury electrolysis, membrane electrolysis or diaphragm electrolysis. Membrane electrolysis is preferred.

In the process according to the first embodiment of the invention, the hydrogen can be used in admixture with another compound. The other compound is usually selected from the group consisting of nitrogen, helium, argon, carbon dioxide, steam, saturated hydrocarbons, and any mixture thereof.

In the process according to the first embodiment of the invention, the resulting mixture containing hydrogen comprises generally at least 10% by volume of hydrogen, usually at least 50% by volume, preferably at least 75% by vol, more preferably at least 90% by volume, still more preferably at least 95% by volume, yet more preferably at least 99% by volume and most preferably at least 99.9% by volume. That mixture comprises generally at most 99.99% of hydrogen by volume. A mixture which consists essentially of hydrogen is also convenient. A mixture which consists of hydrogen is also suitable.

In the process according to the first embodiment of the invention, by zeolite one intends to denote a natural and a synthetic microporous crystalline aluminosilicate, ferrisilicate, gallosilicate, titanosilicate, borosilicate, germanosilicate or silico-alumino-phosphate like SAPO-11. The zeolite is preferably an alumino-silicate.

In the process according to the first embodiment of the invention, an MFI, a MEL, a BEA, a MOR, a FAU, a FER, a MWW, a CHA, a LTA, a ATO or a AEL framework type is as defined for instance in the Database of Zeolite Structures approved by the Structure Commission of the International Zeolite Association (IZA-SC) and available at the internet address http://www.iza-structure.org/databases/.

In the process according to the first embodiment of the invention, the zeolite preferably exhibits a MFI, a MEL, a BEA, a MOR, a FAU or a FER framework type, and more preferably an MFI, a BEA, a MOR or a FAU framework type, and most preferably an MFI framework type.

In the process according to the first embodiment of the invention, by zeolite one also intends to denote mesoporous zeolite as defined for instance in Journal of Catalysis, 2001, 278, 266-275.

In the process according to the first embodiment of the invention, the zeolite might be modified for instance by metal exchange as disclosed for instance in Journal of Catalysis, 1992, 138, 179-194.

In the process according to the first embodiment of the invention, by zeolite being at least partially in the hydrogen form, one intends to denote a zeolite with an alkali metal content of less than 3.5% by weight. The alkali metal is preferably sodium.

In the process according to the first embodiment of the invention, the zeolite is preferably an alumino-silicate selected from the group consisting of ZSM-5, ZSM-11, beta, mordenite, Y and ferrierite, and any mixture thereof. The zeolite is preferably ZSM-5.

In the process according to the first embodiment of the invention, the zeolite has a Si:Al ratio usually higher than equal to 1:1, preferably higher than or equal to 2:1, more preferably higher than or equal to 5:1 and most preferably higher than or equal to 10:1. That Si:Al ratio is usually lower than or equal to 200:1, preferably lower than or equal to 150:1, and more preferably lower than or equal to 100:1.

In the process according to the first embodiment of the invention, the zeolite has an alkali metal content preferably of less than 3.5% by weight, more preferably of less than 3.0% by weight, yet more preferably of less than 2.0% by weight, still more preferably of less than 1.0% by weight, even more preferably of less than 0.1% by weight, most preferably of less than 0.05% by weight, still most preferably lower than or equal to 0.035% by weight and yet most preferably lower than or equal to 0.01% by weight. The alkali metal is preferably sodium.

In the process according to the first embodiment of the invention, a zeolite substantially under H-form, that is to say with an alkali metal content, preferably sodium, of less than 0.035% by weight, is particularly convenient.

In the process according to the first embodiment of the invention, the iridium compound content of the supported catalyst with respect to the zeolite is usually higher than or equal to 0.1% by weight, preferably higher than or equal to 0.2% by weight, more preferably higher than or equal to 0.3% by weight, yet more preferably higher than or equal to 0.5% by weight, still more preferably higher than or equal to 0.8% by weight, most preferably higher than or equal to 1% by weight, still most preferably higher than or equal to 2% by weight and yet most preferably higher than or equal to 3% by weight. That iridium compound content is usually lower than or equal to 20% by weight, preferably lower than or equal to 15% by weight, more preferably lower than or equal to 10% by weight, yet more preferably lower than or equal to 9% by weight and most preferably lower than or equal to 8% by weight.

In the process according to the first embodiment of the invention, the rhenium compound content of the supported catalyst with respect to the zeolite is usually higher than or equal to 0.1% by weight, preferably higher than or equal to 0.2% by weight, more preferably higher than or equal to 0.3% by weight, yet more preferably higher than or equal to 0.5% by weight, still more preferably higher than or equal to 0.8% by weight and most preferably higher than or equal to 1% by weight. That rhenium compound content is usually lower than or equal to 20% by weight, preferably lower than or equal to 15% by weight, more preferably lower than or equal to 10% by weight, yet more preferably lower than or equal to 9% by weight and most preferably lower than or equal to 8% by weight.

In the process according to the first embodiment of the invention, the Ir:Re molar ratio of the supported catalyst is usually higher than or equal to 0.01:1, preferably higher than or equal to 0.05:1, more preferably higher than or equal to 0.1:1, yet more preferably higher than or equal to 0.2:1, still more preferably higher than or equal to 0.3:1, and most preferably higher than or equal to 0.5:1. That Ir:Re ratio is usually lower than or equal to 100:1, preferably lower than or equal to 20:1, more preferably lower than or equal to 10:1, yet more preferably lower than or equal 5:1, still more preferably lower than or equal 3:1 and most preferably lower than or equal to 2:1.

An Ir:Re molar ratio of the supported catalyst higher than or equal to 2:1 and lower than 6:1, and preferably higher than or equal to 3:1 and lower than 5:1, is also particularly convenient.

In the process according to the first embodiment of the invention, at least one part of the iridium compound in the supported catalyst is preferably present in the metallic form. The ratio between the iridium compound present under metallic form and the total iridium compound is preferably higher then or equal to 5%, more preferably higher then or equal to 10%, still more preferably higher then or equal to 50%, yet preferably higher then or equal to 90%, most preferably higher then or equal to 95%, and yet most preferably higher then or equal to 99%. A catalyst where the iridium compound is essentially under metallic form is particularly convenient.

In the process according to the invention, at least one part of the rhenium compound in the supported catalyst is preferably present as a low valence oxide. By low valence oxide, it is meant an oxide wherein the rhenium is at an oxidation state of less than or equal to six but not zero. The ratio between the rhenium compound present under low valence oxide form and the total rhenium is preferably higher then or equal to 5%, more preferably higher then or equal to 10%, still more preferably higher then or equal to 50%, yet preferably higher then or equal to 90%, most preferably higher then or equal to 95%, and yet most preferably higher then or equal to 99%. A catalyst where the rhenium compound is essentially under low valence oxide form is particularly convenient.

Said content of iridium compound under metallic form and said rhenium compound under low valence oxide form can be determined by X-ray diffraction analysis, Temperature programmed Reduction and EXFAS as disclosed in Nagawa et al. Journal of Catalysis: 272, 2010, 191-194 and by X-ray Photoelectron Spectroscopy.

In the process according to the first embodiment of the invention, the zeolite may contain elements other than iridium and rhenium. Such element is preferably selected from the group consisting of rhodium, platinum, vanadium, lanthanum, cerium, tungsten and any mixture thereof.

In the process according to the first embodiment of the invention, the supported catalyst may contain at least one component other than iridium and rhenium supported on a zeolite, for instance a binder used for shaping the catalyst. The other component is preferably selected from the group consisting of silica, alumina, silica-alumina, clay, boehmite, silica-magnesia, kaolin and any mixture thereof.

In the process according to the first embodiment of the invention, the catalyst preferably consists of at least one iridium compound and at least one rhenium compound, both compounds being supported on a zeolite.

In the process according to the invention, the catalyst is usually in a form selected from the group consisting of rings, beads, spheres, saddles, pellets, tablets, extrudates, granules, crushed, flakes, honeycombs, filaments, cylinders, polygons and any mixture thereof, or in powder form. The powder form is a preferred form. The extrudates form is another preferred form.

The process according to the first embodiment of the invention can be carried out according to any mode of operation. The mode of operation can be continuous or discontinuous. By continuous mode of operation, one intends to denote a mode of operation where the glycerol and hydrogen are added continuously in the process, and the 1,3-propanediol is continuously withdrawn from the process. Any other mode of operation is considered as discontinuous. The continuous mode is preferred. The discontinuous mode is also convenient

In the process according to the first embodiment of the invention, the reaction may be carried out in a gas phase or in at least one liquid phase, preferably in at least one liquid phase.

In the process according to the first embodiment of the invention, the reaction can be carried out in the presence of at least one acid. The acid can be a inorganic acid, an organic acid or a mixture thereof. The organic acid can be for instance a sulfonic acid, like methane sulfonic acid, benzene sulfonic acid or toluene sulfonic acid. The acid is preferably an inorganic acid and most preferably sulfuric acid.

In the process according to the first embodiment of the invention, when the reaction is carried out in at least one liquid phase, and in the presence of an acid, preferably sulfuric acid, the content of the acid in the at least one liquid phase is usually higher than or equal to 0.01 g/kg of liquid phase, more preferably higher than or equal to 0.05 g/kg and most preferably higher than or equal to 0.1 g/kg. This content of acid is usually lower than or equal to 50 g/kg of liquid phase, more preferably lower than or equal to 10 g/kg and most preferably lower than or equal to 2 g/kg. The reaction is preferably carried out in the absence of acid that is to say at a content of acid in the liquid phase lower than or equal to 1 g/kg.

In the process according to the invention, the reaction can be carried out in the presence of water.

In the process according to the first embodiment of the invention, when the reaction is carried out in at least one liquid phase, and in the presence of water, the content of water in the at least one liquid phase is usually higher than or equal to 1 g/kg of liquid phase, preferably higher than or equal to 2 g/kg, more preferably higher than or equal to 5 g/kg, yet more preferably higher than or equal to 10 g/kg, still more preferably higher than or equal to 50 g/kg, most preferably higher than or equal to 100 g/kg, yet most preferably higher than or equal to 150 g/kg and still most preferably higher than or equal to 200 g/kg. That content of water is usually lower than or equal to 999 g/kg, preferably lower than or equal to 950 g/kg, more preferably lower than or equal to 900 g/kg, yet more preferably lower than or equal to 850 g/kg, still more preferably lower than or equal to 825 g/kg and most preferably lower than or equal to 800 g/kg. A water content lower than or equal to 250 g/kg is also suitable.

In the process according to the first embodiment of the invention, when the reaction is carried out in at least one liquid phase, it may be carried out in the absence or in the presence of a solvent. The solvent may be selected from the group consisting of inert inorganic solvent, inert organic solvent, and combinations thereof. Examples of inert inorganic solvents are water, supercritical carbon dioxide, and inorganic ionic liquids. Examples of inert organic solvents are alcohols, ethers, saturated hydrocarbons, esters, perfluorinated hydrocarbons, nitriles, amides and any mixture thereof. Examples of alcohols are methanol, ethanol, 1-propanol, 2-propanol, ethylene glycol, 1,2-propanediol and 1,3-propanediol. Examples of ethers are diethylene glycol, dioxane, tetrahydrofuran, ethylene glycol monomethyl ether, ethylene glycol monoethyl ether, ethylene glycol dimethyl ether, diethylene glycol monomethyl ether and diethylene glycol dimethyl ether. An example of saturated hydrocarbons is cyclohexane. An example of esters is ethyl acetate. Examples of perfluorinated hydrocarbons are perfluorinated alkane such as perfluorinated hexane, heptane, octane, nonane, cyclohexane, methylcyclohexane, dimethylcyclohexane or trimethylcyclohexane, perfluoroethers like Galden® HT from Solvay Solexis, perfluorotetrahydrofurane or perfluoroamine like Fluorinert™ FC from 3M. An example of nitriles is acetonitrile. An example of amides is dimethylformamide.

In the process according to the first embodiment of the invention, the reaction is preferably carried out in the presence of a solvent. The solvent is preferably water.

In the process according to the first embodiment of the invention, when the reaction is carried out in the presence of at least one liquid phase and in the presence of a solvent, preferably water, the content of the solvent in the at least one liquid phase is usually higher than or equal to 1 g/kg of liquid phase, preferably higher than or equal to 2 g/kg, more preferably higher than or equal to 5 g/kg, yet more preferably higher than or equal to 10 g/kg, still more preferably higher than or equal to 50 g/kg, most preferably higher than or equal to 100 g/kg, yet most preferably higher than or equal to 150 g/kg and still most preferably higher than or equal to 200 g/kg. That content of solvent is usually lower than or equal to 999 g/kg, preferably lower than or equal to 950 g/kg, more preferably lower than or equal to 900 g/kg, yet more preferably lower than or equal to 850 g/kg, still more preferably lower than or equal to 825 g/kg and most preferably lower than or equal to 800 g/kg.

In the process according to the first embodiment of the invention, when the reaction is carried out in at least one liquid phase and in the presence of water, the at least one liquid phase preferably contains less than 100 g of sulfuric acid per kg liquid phase, more preferably less than 50 g of sulphuric acid, yet more preferably less than 10 g of sulphuric acid, still more preferably less than 5 g of sulphuric acid and is preferably substantially free of sulfuric acid, that is to say contains less than 1 g of sulfuric acid per kg of liquid phase.

In the process according to the first embodiment of the invention, the reaction may also be carried out solvent-less, i.e. for a content of the solvent, excluding water, in the liquid phase lower than 1 g/kg.

In the process according to the first embodiment of the invention, the reaction may be suitably carried out in a gas phase.

In addition to hydrogen and to the glycerol the gas phase may also contain a diluent. The diluent may be a compound as described as solvent here above or a compound as used in admixture with hydrogen as described here above. The diluent is preferably water, more preferably steam.

The process according to the first embodiment of the invention can be carried out in reaction apparatuses made of or coated with materials which are suitable for hydrogenation under pressure, resistant in the presence of corrosive compounds under the reaction conditions. Suitable materials can be selected from the group consisting of glass, enamel, enameled steel, graphite, impregnated graphite, like for example graphite impregnated with a perfluorinated polymer such as polytetrafluoroethylene, or graphite impregnated with a phenolic resin, polyolefins, like for instance polyethylene or polypropylene, fluorinated polymers, like perfluorinated polymers such as for instance polytetrafluoroethylene, poly(perfluoropropylvinylether), copolymers of tetrafluoroethylene and hexafluoropropylene, and like partially fluorinated polymers such as for instance poly(vinylidene fluoride), polymers comprising sulphur, like for instance polysulphones or polysulphides, metals, like for instance tantalum, titanium, copper, gold, silver, nickel and molybdenum, and metal alloys, like for instance alloys containing nickel, such as Hastelloy B, Hastelloy C, alloys containing molybdenum, such as Inconel 600, Inconel 625 or Incoloy 825.

The materials may be used within the mass, or in the form of cladding, or else by means of any coating process. Enameled steel is particularly convenient. Glass-lined apparatuses are also convenient.

In the process according to the first embodiment of the invention, the reaction is carried out at a temperature preferably higher than or equal to 70° C., more preferably higher than or equal to 80° C., yet more preferably higher than or equal to 90° C. and most preferably higher than or equal to 100° C. That temperature is preferably lower than or equal to 300° C., more preferably lower than or equal to 200° C., yet more preferably lower than or equal to 180° C. and most preferably lower than or equal to 150° C.

In the process according to the first embodiment of the invention, the reaction is preferably carried out at a hydrogen partial pressure preferably higher than or equal to 1 bar absolute (1 bara), more preferably higher than or equal to 5 bara and most preferably higher than or equal to 10 bara. That hydrogen partial pressure is preferably lower than equal to 200 bara, more preferably lower than equal to 150 bara and most preferably lower than equal to 120 bara.

In the process according to the first embodiment of the invention carried out under continuous mode, especially when at least one liquid phase is present, the residence time which is the ratio of the volume of the liquid phase to the flow rate by volume of the reactants, depends on the reaction rate, on the hydrogen partial pressure, on the temperature, on the thoroughness with which the reaction mixture is mixed, and on the activity and concentration of the supported catalyst. This residence time is usually higher than or equal to 5 minutes, often higher than or equal to 15 minutes, frequently higher than or equal to 30 minutes and in particular higher than or equal to 60 minutes. This residence time is usually lower than or equal to 25 hours, often lower than or equal to 20 hours, frequently lower than or equal to 10 and in particular lower than or equal to 5 hours.

In the process according to the first embodiment of the invention carried out under continuous mode, especially when a gas and a liquid phase are present, the residence time for the liquid phase, which is the ratio of the volume of the reactor volume to the flow rate by volume of the liquid phase, is usually higher than or equal to 5 minutes, often higher than or equal to 15 minutes, frequently higher than or equal to 30 minutes and in particular higher than or equal to 60 minutes. This residence time is usually lower than or equal to 25 hours, often lower than or equal to 10 hours and frequently lower than or equal to 5 hours.

In the process according to the first embodiment of the invention carried out under continuous mode, especially when a gas and a liquid phase are present, the residence time for the gas phase, which is the ratio of the volume of the reactor volume to the flow rate by volume of the gas phase, is usually higher than or equal to 1 second, often higher than or equal to 5 seconds, frequently higher than or equal to 10 seconds and in particular higher than or equal to 30 seconds. This residence time is usually lower than or equal to 10 minutes, often lower than or equal to 5 minutes and frequently lower than or equal to 2 minutes.

In the process according to the first embodiment of the invention carried out under continuous mode, especially when the reaction is carried out in the a gas phase, the residence time for the gas phase, which is the ratio of the volume of the reactor volume to the flow rate by volume of the gas phase, is usually higher than or equal to 1 second, often higher than or equal to 5 seconds, frequently higher than or equal to 10 seconds and in particular higher than or equal to 30 seconds. This residence time is usually lower than or equal to 10 minutes, often lower than or equal to 5 minutes and frequently lower than or equal to 2 minutes.

In the process according to the first embodiment of the invention carried out under discontinuous mode, the reaction time required for the process according to the invention depends on the reaction rate, on the hydrogen partial pressure, on the temperature, on the thoroughness with which the reaction mixture is mixed, and on the activity and concentration of the supported catalyst. The required reaction time is usually higher than or equal to 5 minutes, often higher than or equal to 15 minutes, frequently higher than or equal to 30 minutes, in particular higher than or equal to 60 minutes, and more specifically higher than or equal to 160 min. This residence time is usually lower than or equal to 25 hours, often lower than or equal to 20 hours, frequently lower than or equal to 10 and in particular lower than or equal to 5 hours.

In the process according to the first embodiment of the invention carried out under discontinuous mode, especially when a gas and a liquid phase are present, the reaction time for the liquid phase is usually higher than or equal to 5 minutes, often higher than or equal to 15 minutes, frequently higher than or equal to 30 minutes, in particular higher than or equal to 60 minutes, and more specifically higher than or equal to 180 min. This reaction time is usually lower than or equal to 25 hours, often lower than or equal to 10 hours and frequently lower than or equal to 5 hours.

In the process according to the first embodiment of the invention carried out under discontinuous mode, especially when a gas and a liquid phase are present, the reaction time for the gas phase is usually higher than or equal to 1 second, often higher than or equal to 5 seconds, frequently higher than or equal to 10 seconds and in particular higher than or equal to 30 seconds. This reaction time is usually lower than or equal to 10 minutes, often lower than or equal to 5 minutes and frequently lower than or equal to 2 minutes.

In the process according to the first embodiment of the invention, especially when the reaction is carried out continuously in the a gas phase, the ratio between the flow rates of hydrogen and glycerol is usually higher than or equal to 0.1 mol/mol, often higher than or equal to 0.5 mol/mol, frequently higher than or equal to 1 mol/mol seconds and in particular higher than 1 mol/mol. This ratio is usually lower than or equal to 100 mol/mol, often lower than or equal to 50 mol/mol, frequently lower than or equal to 20 mol/mol and in particular lower than or equal to 10 mol/mol.

The process according to the first embodiment of the invention may be carried out in any type of reactor. The reactor, especially when the reaction is carried out in the presence of a catalyst, may be selected from the group consisting of a slurry reactor, a fixed bed reactor, a trickle bed reactor, a fluidized bed reactor, and combinations thereof. A slurry reactor or a trickle bed reactor is particularly convenient when the reaction is carried out in a least one liquid phase. A trickle bed reactor is more particularly suitable. A trickle bed reactor fed at co-current by hydrogen and the glycerol is very particularly convenient. A fixed bed reactor, a fluidized bed reactor, a moving bed reactor are particularly convenient when the reaction is carried out in the gas phase. A reactor containing the catalyst as a packing of honeycomb structures is well suited when the reaction is carried out in the gas phase.

In a simple way of carrying out the process according to the first embodiment of the invention, the process can be carried out discontinuously, in the following manner: an autoclave which is provided with a stirring or mixing unit and which can be thermostated is charged, in a suitable manner, with glycerol to be hydrogenated, the catalyst and a possible solvent. Thereafter, hydrogen is forced in until the desired pressure is reached, and the mixture is heated to the chosen reaction temperature while mixing thoroughly. The course of the reaction can be readily monitored by measuring the amount of hydrogen consumed, which is compensated by feeding in further hydrogen. The hydrogenation is complete when hydrogen is no longer consumed, and the amount of hydrogen consumed corresponds approximately to the theoretically required amount of hydrogen.

The mixture present after the hydrogenation can be worked up, for example, as follows: when the hydrogenation is complete, the reaction vessel is cooled, the pressure is let down and the catalyst is filtered off and rinsed with the possible solvent used, and the possible solvent used is then removed under reduced pressure or under atmospheric pressure. The crude product which remains can likewise be purified further by distillation under reduced pressure or under atmospheric pressure. When high-boiling solvents are used, it is also possible first to distil off the propanediol. When no solvent is used the mixture can be submitted directly to a distillation under reduced pressure or under atmospheric pressure. The recovered catalyst e.g by filtration can be reused in the discontinuous process as such or after reactivation by a physico-chemical treatment.

In another way of carrying out the process according to the first embodiment of the invention, the process can be carried out continuously, in the following manner: a vertical cylindrical reactor which can be thermostated is charged with the catalyst provided in a suitable shape in order to obtain a fixed bed of catalyst in the reactor. The reactor is fitted on its top part with inlet ports to feed the glycerol to be hydrogenated, neat or dissolved in a solvent and hydrogen, on its bottom part with an outlet port to recover the reaction mixture, with a regulation pressure device, and a vessel, to recover the reaction mixture and separate the liquid phase and the gas phase. Thereafter, hydrogen is forced continuously in the reactor until the desired pressure is reached, then the reactor is heated to the chosen reaction temperature, the glycerol, neat or dissolved in a solvent, is forced continuously in the reactor and the reaction mixture is collected continuously in the recovery vessel. The extent of the reaction can be readily monitored by analyzing the composition of the liquid separated in the collection vessel. The liquid mixture present in the recovery vessel can be worked up, for example, by distillation under reduced pressure or under atmospheric pressure.

When the process according to the first embodiment of the invention is carried out continuously, a continuous process purge of gaseous by-products of the reaction or of gaseous contaminants of raw materials may be present.

Gas chromatography is usually used for assessing the content of the organic compounds in samples withdrawn at various stages of the process.

The present invention also relates in a second embodiment to a process for making a polymer selected from the group consisting of a polyether, a polyurethane, a polyester, and any mixture thereof, comprising obtaining 1,3-propanediol by reacting by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form, and further using said 1,3-propanediol as raw materials.

The process for making the polyether comprises obtaining 1,3-propanediol by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form, and further submitting said 1,3-propanediol to a reaction with at least one compound selected from the group consisting of a halogenated organic compound, an organic epoxide, an alcohol, or any mixture thereof.

The process for making the polyurethane comprises obtaining 1,3-propanediol by reacting by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form, and further submitting said 1,3-propanediol to a reaction with a polyisocyanate, preferably a diisocyanate.

In the process for making the polymer according to the invention, the polymer is preferably a polyester.

The present invention therefore also relates to a process for making a polyester comprising obtaining 1,3-propanediol by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form, and further submitting said 1,3-propanediol to a reaction with a carboxylic acid and/or a carboxylic acid ester.

The features mentioned above for the process for manufacturing the 1,3-propanediol are applicable for obtaining the 1,3-propanediol used for making the polymer, preferably the polyester.

In the process for making a polyester according to the invention, the carboxylic acid is preferably a polycarboxylic acid, more preferably a dicarboxylic acid. The polycarboxylic acid is preferably selected from the group consisting of an aliphatic saturated or unsaturated, an aromatic, an alkylaromatic saturated or unsaturated, a heteroaromatic, an alkylheteroaromatic saturated or unsaturated, or any mixture thereof.

The preferred aliphatic dicarboxylic acid contains from 2 to 16 carbon atoms are more preferably selected from the group consisting of oxalic acid, malonic acid, succinic acid, glutaric acid, adipic acid, sebacic acid, azelaic acid and any mixture thereof.

The preferred unsaturated dicarboxylic acid is selected from the group consisting of fumaric acid, maleic acid, and any mixture thereof.

The preferred aromatic is selected from the group consisting of o-phthalic acid, m-phthalic acid, p-phthalic acid (terephthalic acid), naphthalene dicarboxylic acid, and any mixture thereof. The preferred aromatic carboxylic acid is more preferably terephthalic acid.

The preferred alkyl aromatic is selected from the group consisting of 4-methylphthalic acid, 4-methylphthalic acid, and any mixture thereof.

The preferred unsaturated dicarboxylic aromatic acid is selected from the group consisting of vinyl phthalic acids, and any mixture thereof.

The preferred heteroaromatic acid is selected from the group consisting of furano dicarboxylic acids, and any mixture thereof, and is preferably 2,5-furano dicarboxylic acid.

In the process for making a polyester according to the invention, the carboxylic acid ester is preferably an ester of the above cited carboxylic acid, preferably a methyl, or ethyl ester. The preferred ester is selected from the group consisting of an ester of terephthalic acid, an ester of furano dicarboxylic acid, and any mixture thereof. The ester is more preferably a terephthalic acid ester, and most preferably dimethyl terephthalate.

The production of the polyester is described, for example, in Ullmann's Encyclopedia of Industrial Chemistry, Horst Köpnick, Manfred Schmidt, Wilhelm Brügging, Jörn Miter and Walter Kaminsky, Published Online: 15 Jun. 2000, DOI: 10.1002/14356007.a21_{—}227, pages 623-649.

The polymer, preferable a polyester, obtained according to the process of the invention usually exhibits a ¹⁴C/¹²C higher than or equal to 0.33 10⁻¹², often higher than or equal to 0.5 10⁻¹², frequently higher than or equal to 0.75 10⁻¹², in many case higher than or equal to 1.0 10⁻¹² and in particular higher than or equal to 1.1 10⁻¹².

In a further embodiment, the invention also relates to a polyester exhibiting a ¹⁴C/¹²C higher than or equal to 0.33 10⁻¹², often higher than or equal to 0.5 10⁻¹², frequently higher than or equal to 0.75 10⁻¹², in many case higher than or equal to 1.0 10⁻¹² and in particular higher than or equal to 1.1 10⁻¹².

The polyester is preferably obtainable by reacting 1,3-propanediol obtained by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form, and further submitting said 1,3-propanediol to a reaction with a carboxylic acid and/or a carboxylic acid ester, as described here above.

The polyester is more preferably obtained by reacting 1,3-propanediol obtained by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form, and further submitting said propanediol to a reaction with a carboxylic acid and/or a carboxylic acid ester, as described here above.

The present invention also relates in a third embodiment to a process for making a polyester fiber comprising obtaining 1,3-propanediol by reacting 1,3-propanediol obtained by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form, further submitting said 1,3-propanediol to a reaction with a carboxylic acid and/or a carboxylic acid ester to obtain a polyester and further converting said polyester in to a fiber.

The methods for measuring the ¹⁴C content are precisely described in standards ASTM D 6866 (notably D 6866-06 and D 6866-08) and in standards ASTM 7026 (notably D 7026-04). The method preferably used is described in standard ASTM D6866-08.

The features mentioned above for the process for manufacturing the 1,3-propanediol and for the process for making the polyester are applicable for obtaining the 1,3-propanediol and the polyester used for making the polyester fiber.

The production of the polyester fibers is described, for example, in Ullmann's Encyclopedia of Industrial Chemistry, Helmut Sattler and Michael Schweizer, Published Online: 15 Oct. 2011, DOI: 10.1002/14356007.o10_o01, pages 1 to 34.

The polyester fibers have numerous applications and can be used in tires, rope, cordage, sewing thread, seat belts, hoses, webbing, coated fabrics, carpets, apparel, home fashions, upholstery, medical, interlinings, filtration, fiberfill, high-loft, roofing, geotextiles, and substrates, for instance.

In fourth embodiment, the invention also relates to a process for preparing a supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is partially in the hydrogen form, comprising co-impregnating said zeolite with an aqueous composition containing at least one iridium compound and at least one rhenium compound, drying said co-impregnated zeolite, calcining said dried co-impregnated zeolite and further treating the said calcined co-impregnated zeolite under hydrogen in water at a temperature higher than or equal to 150° C. and lower than or equal to 350° C.

All the features disclosed hereabove for the iridium and rhenium compounds supported on the zeolite are applicable to the process for preparing the catalyst.

The co-impregnation can be carried out according to any know technique of impregnation as described in “Applied Catalysis A: General, 1995, 133, 281-292”. Incipient-wetness co-impregnation is preferred. Any solvent can be used provided that the iridium and rhenium precursors are soluble therein. Water is a preferred solvent. Any iridium and rhenium precursors can be used provided that they are soluble in the solvent used. Chloroiridic acid and ammonium perrhenate are preferred, in particular when the solvent used is water.

The drying of the co-impregnated zeolite can be carried out according to any known technique. Drying is carried out at a temperature preferably higher than or equal to 50° C., more preferably higher than or equal to 70° C., and most preferably higher than or equal to 90° C. This drying temperature is preferably lower than or equal to 180° C., more preferably lower than or equal to 150° C., and most preferably lower than or equal to 130° C. Drying is carried out at a pressure preferably higher than or equal to 400 mbar absolute, more preferably higher than or equal to 600 mbara, and most preferably higher than or equal to 800 mbara. This drying pressure is preferably lower than or equal to 1800 mbara, more preferably lower than or equal to 1500 mbara, and most preferably lower than or equal to 1200 mbara.

The calcination of the dried co-impregnated zeolite can be carried out according to any known technique. Calcination is carried out at a temperature preferably higher than or equal to 300° C., more preferably higher than or equal to 350° C., and most preferably higher than or equal to 400° C. This calcination temperature is preferably lower than or equal to 700° C., more preferably lower than or equal to 650° C., and most preferably lower than or equal to 600° C.

The treatment under hydrogen is carried out in water at a temperature preferably higher than or equal to 150° C., more preferably higher than or equal to 160° C., and most preferably higher than or equal to 170° C. This treatment temperature is preferably lower than or equal to 325° C., more preferably lower than or equal to 275° C., and most preferably lower than or equal to 250° C.

The examples below are intended to illustrate the invention without, however, limiting it.

1. Catalyst Preparation

The supported catalysts were prepared by co-impregnating supports with an aqueous solution (5 g H₂O/g support) of H₂IrCl₄.xH₂O and NH₄ReO₄. The impregnated support was stirred, heated to 80° C. and kept at this temperature for 3 hours. The impregnated supports were dried at 110° C. overnight at a pressure of about 1 bara, and the dried supports were then calcined at 500° C. in static air for 3 hours. The following zeolites have been used as support: HZSM-5 (90) (0.03 wt % Na₂O), HZSM-5 (59) (0.15 wt % Na₂O), NH₄ZSM-5 (28) (0.02 wt % Na₂O) and NH₄-beta (25) (0.04 wt % Na₂O) MOR (40) provided by Süd-Chemie, and HY (80) (0.03 wt % Na₂O), HY (60) (0.03 wt % Na₂O), HY (12) (0.05 wt % Na₂O) and SAPO-11 obtained from Zeolyst, FER (20) obtained from Alfa Aesar. The numbers between brackets represent the molar ratio of SiO₂/Al₂O₃ for the corresponding zeolites. The zeolites under NH₄-form were further treated by calcination at 550° C. for 6 hours to obtain the corresponding H-form zeolites. A silica support has also been used (G-6, Fuji Silysia Chemical Ltd). The loading contents of both Ir and Re after calcination were measured by ICP-OES and are presented expressed as elemental Ir and Re in Table 1 here below.

The catalyst are identified in the following Table 1.

TABLE 1

| Catalyst no | Ir—Re (wt %) | Zeolite as received |
|---|---|---|
| 1 | 4-4 | HZSM5 (90) |
| 2 | 4-4 | HZSM5 (59) |
| 3 | 4-2 | HZSM5 (59) |
| 4 | 4-6 | HZSM5 (59) |
| 5 | 4-4 | NH₄ZSM5 (28) |
| 6 | 4-4 | NH₄beta (25) |
| 7 | 4-4 | HY (80) |
| 8 | 2-4 | HY (80) |
| 9 | 4-4 | HY (60) |
| 10 | 4-4 | HY (12) |
| 11 | 4-4 | MOR (40) |
| 12 | 4-4 | FER (20) |
| 13 | 4-4 | SAPO-11 |
| 14 | 4-4 | SiO₂ |

2. Catalyst Evaluation

The hydrogenation of glycerol has been carried out in a glass vessel fitted in a 100 ml Hastelloy (C-22) autoclave.

The catalyst was first pretreated as follows. The desired quantity of the calcined impregnated support, a magnetic stirring and water were placed in the glass vessel. The autoclave was sealed and purged with nitrogen (N₂, Air Product, 99.998%) and then hydrogen (H₂, Air Product, 99.9995%) for several times, then heated to 200° C. under a H₂ pressure of 80 bara for 1 hour. The autoclave was then cooled down, the hydrogen pressure was then released, and the autoclave was opened. Glycerol, water and optionally sulphuric acid (Aldrich, 95-98%) were then added to the glass vessel so that to reach 10 g of the glycerol (VWR, 99.5%)-water (Milli-Q water) mixture and subsequently, the autoclave was closed, purged with nitrogen and hydrogen again, and then heated to the desired temperature of 120° C. Meanwhile, the hydrogen pressure was increased to 80 bara and stirring was set at 750 rpm. The time zero for the reaction was defined as the time at which the stirring was started. During the reaction, the pressure was always maintained at 80 bara. After an appropriate reaction time, the reaction was stopped and the autoclave was cooled down. The liquid product was separated by using polypropylene filter and then analyzed by gas chromatography. The mass balance was calculated for each test and it was found higher than 95% in most cases.

The conditions for carrying out examples 1 to 20 are summarized in Table 2.

The final water quantity corresponds to the water present in the glass vessel before stirring was initiated

TABLE 2

| | Catalyst calcined | Pretreatment | | Reaction | | | | | |
|---|---|---|---|---|---|---|---|---|---|
| Example | N^{o} | catalyst (g) | water (g) | H₂SO₄ (g) | final water (g) | glycerol (g) | T (° C.) | P (bara) | Time (h) |
| 1 | 2 | 0.15 | 3 | 0.003 | 9 | 1 | 120 | 80 | 5 |
| 2 | 2 | 0.15 | 3 | — | 9 | 1 | 120 | 80 | 5 |
| 3 | 3 | 0.15 | 3 | — | 9 | 1 | 120 | 80 | 5 |
| 4 | 4 | 0.15 | 3 | — | 9 | 1 | 120 | 80 | 5 |
| 5 | 5 | 0.15 | 3 | — | 9 | 1 | 120 | 80 | 5 |
| 6 | 2 | 0.15 | 3 | — | 9 | 1 | 140 | 80 | 3 |
| 7 | 2 | 0.5 | 2 | — | 2 | 8 | 120 | 80 | 5 |
| 8 | 1 | 0.15 | 3 | — | 9 | 1 | 120 | 80 | 5 |
| 9 | 1 | 0.3 | 2 | — | 2 | 8 | 120 | 80 | 5 |
| 10 | 6 | 0.15 | 3 | — | 9 | 1 | 120 | 80 | 5 |
| 11 | 7 | 0.15 | 3 | — | 9 | 1 | 120 | 80 | 5 |
| 12 | 7 | 0.15 | 3 | — | 8 | 2 | 120 | 80 | 5 |
| 13 | 7 | 0.3 | 2 | — | 2 | 8 | 120 | 80 | 4 |
| 14 | 8 | 0.3 | 2 | — | 2 | 8 | 120 | 80 | 5 |
| 15 | 9 | 0.15 | 3 | — | 8 | 2 | 120 | 80 | 5 |
| 16 | 10 | 0.15 | 3 | — | 8 | 2 | 120 | 80 | 5 |
| 17 | 11 | 0.15 | 3 | — | 9 | 1 | 120 | 80 | 5 |
| 18 | 12 | 0.15 | 3 | — | 9 | 1 | 120 | 80 | 5 |
| 19 | 13 | 0.15 | 3 | — | 8 | 2 | 120 | 80 | 5 |
| 20 | 14 | 0.15 | 3 | — | 9 | 1 | 120 | 80 | 5 |

Examples 1 to 19 are according to the invention and example 20 is not according to the invention.

The results of the tests are summarized in Table 3 here below.

The glycerol conversion and the selectivity of the various products have been calculated as follows, except for examples 7, 9, 13 and 14:

Conversion=100×[(number of mole of glycerol introduced−number of mole of glycerol recovered at the end of reaction)/(number of mole of glycerol introduced)].

Selectivity product=100×[(number of mole of product recovered at the end of reaction)/(number of mole of glycerol introduced−number of mole of glycerol recovered at the end of reaction)].

The glycerol conversion and the selectivity of the various products for examples 7, 9, 13 and 14 have been calculated as follows:

Conversion=100×[(sum of number of mole of 1,3-propanediol, 1,2-propanediol, 1-propanol and 2-propanol recovered at the end of reaction)/(number of mole of glycerol introduced)].

Selectivity product=100×[(number of mole of product recovered at the end of reaction)/(sum of number of mole of 1,3-propanediol, 1,2-propanediol, 1-propanol and 2-propanol recovered at the end of reaction)].

The quantities of iridium and rhenium leached from the catalyst at the end of the reaction are also presented in Table 3 for some catalysts. Those quantities are expressed as the ratio in percent between the iridium and rhenium found in the liquid product after separation by filtration and the iridium and rhenium initially present in the catalyst quantity used before the reaction, the quantities being expressed as elemental iridium or rhenium. The iridium and rhenium quantities in the liquid product after separation by filtration have been obtained by ICP-OES (Inductively coupled plasma optical emission spectroscopy).

TABLE 3

| | Conversion of | | | | | | |
|---|---|---|---|---|---|---|---|
| | Glycerol | Product selectivity (% mol) | | | | Leached metals | |
| Example | (% mol) | 1,3-Propanediol | 1,2-propanediol | 1-propanol | 2-propanol | Ir (%) | Re (%) |
| 1 | 22.6 | 62 | 17.1 | 12.4 | 8.0 | n.a. | n.a. |
| 2 | 27.6 | 59.4 | 14 | 14.5 | 8.6 | n.a. | n.a. |
| 3 | 27.5 | 55.4 | 15.8 | 15.1 | 8.6 | n.a. | n.a. |
| 4 | 26.3 | 58.1 | 16.1 | 15.8 | 11.3 | n.a. | n.a. |
| 5 | 11.8 | 59.3 | 25.7 | 6.2 | 8.1 | n.a. | n.a. |
| 6 | 33 | 49.5 | 13.1 | 19 | 4.9 | n.a. | n.a. |
| 7 | 11.7 | 69.8 | 9 | 16 | 5.2 | 0.025 | 0.075 |
| 8 | 32.3 | 58.2 | 12.4 | 19.8 | 9.7 | 0.050 | 0.15 |
| 9 | 11 | 72.6 | 8.2 | 14.3 | 4.9 | n.a. | n.a. |
| 10 | 12.5 | 58.3 | 16.9 | 9.5 | 7.1 | n.a. | n.a. |
| 11 | 49.4 | 45.4 | 8.4 | 21.8 | 7.0 | n.a. | n.a. |
| 12 | 30 | 56.4 | 10.7 | 21.4 | 7.1 | n.a. | n.a. |
| 13 | 11.6 | 65.9 | 7.8 | 21.2 | 5.1 | n.a. | n.a. |
| 14 | 5.8 | 49.8 | 8 | 34 | 8.2 | n.a. | n.a. |
| 15 | 25.7 | 59.1 | 11.6 | 15.8 | 7.5 | n.a. | n.a. |
| 16 | 32.6 | 58.3 | 12 | 21.5 | 7.8 | n.a. | n.a. |
| 17 | 24.2 | 57.4 | 17.1 | 17.5 | 7.9 | n.a. | n.a. |
| 18 | 19.9 | 55.2 | 20.6 | 15 | 9 | n.a. | n.a. |
| 19 | 1.4 | 58.2 | 20.8 | 10.3 | 10.5 | n.a. | n.a. |
| 20 | 25.3 | 50.5 | 21 | 15.6 | 8.1 | 0.05 | 0.5 |

n.a.: not analyzed.

### EXAMPLE 21

The procedure for the catalyst evaluation disclosed above has been used. 4.5 g of catalyst n° 1 have been used. 120 g of glycerol and 30 g of water have been added to the catalyst. The reaction has been carried out for 14 h, at 120° C. and 80 bara of hydrogen. The conversion of glycerol and the selectivities of the various products of reaction have been recorded at various times of reaction and they are presented in Table 4 here below.

TABLE 4

| | Glycerol | Selectivities (mol %) | | | |
|---|---|---|---|---|---|
| Time | conversion | 1,3- | 1,2- | 1- | 2- |
| (min) | (mol %) | propanediol | propanediol | propanol | propanol |
| 120 | 0.7 | 71.6 | 10.3 | 11.6 | 5.8 |
| 240 | 10.1 | 69.4 | 9.3 | 14.2 | 6.1 |
| 360 | 14.1 | 68.5 | 8.9 | 15.8 | 6.2 |
| 540 | 20.7 | 67.7 | 8.9 | 17 | 6.0 |
| 840 | 28 | 66.5 | 8.4 | 18.7 | 5.7 |

1. A process for manufacturing 1,3-propanediol by reacting glycerol with hydrogen in the presence of a supported catalyst, the supported catalyst comprising at least one iridium compound and at least one rhenium compound, both compounds being supported on a zeolite, wherein the zeolite exhibits an MFI, a MEL, a BEA, a MOR, a FAU, a FER, a MWW, a CHA, a LTA, a ATO or a AEL framework type, and wherein the said zeolite is at least partially in the hydrogen form.

2. The process according to claim 1, wherein the zeolite exhibits an MFI, a MEL, a BEA, a MOR, a FAU or a FER framework type.

3. The process according to claim 2, wherein the zeolite is an alumino-silicate selected from the group consisting of ZSM-5, ZSM-11, beta, mordenite, Y, ferrierite, and any mixture thereof.

4. The process according to claim 3, wherein the zeolite is ZSM-5.

5. The process according to claim 3, wherein the zeolite is Y.

6. The process according to claim 3, wherein the zeolite is beta.

7. The process according to claim 3, wherein the zeolite is mordenite.

8. (canceled)

9. The process according to claim 3, wherein the zeolite is ferrierite.

10. The process according to claim 1, wherein the zeolite is an alumino-silicate and wherein the zeolite has a Si:Al ratio higher than or equal to 1:1, and lower than or equal to 200:1

11-17. (canceled)

18. The process according to claim 1, wherein the zeolite has an alkali metal content of less than 3.5% by weight

19-20. (canceled)

21. The process according to claim 18, wherein the alkali metal content of the zeolite is of less than 1% by weight and wherein the alkali metal is sodium.

22-25. (canceled)

26. The process according to claim 21, wherein the zeolite is substantially under H-form.

27. The process according to claim 1, wherein the supported catalyst exhibits at least one of the following features:
(i) the iridium compound content of the supported catalyst with respect to the zeolite is higher than or equal to 0.1% by weight and lower than or equal to 10% by weight;
(ii) the rhenium compound content of the supported catalyst with respect to the zeolite is higher than or equal to 0.1% by weight and lower than or equal to 10% by weight.

28-39. (canceled)

40. The process according to claim 1, wherein the reaction is carried out under at least one of the following conditions:
(a) at a temperature higher than or equal to 80° C. and lower than or equal to 300° C.;
(b) at a hydrogen partial pressure higher than or equal to 1 bar absolute and lower than equal to 200 bar absolute.

41. The process according to claim 1, wherein the reaction is carried out in at least one liquid phase, under at least one of the following conditions:
(A) in the presence of water, the content of water in the at least one liquid phase being higher than or equal to 1 g/kg of liquid phase and lower than or equal to about 999 g/kg of liquid phase;
(B) in the presence of sulfuric acid, the content of sulfuric acid in the at least one liquid phase being higher than or equal to 0.01 g/kg of liquid phase and lower than or equal to about 50 g/kg of liquid phase.

42. (canceled)

43. The process according to claim 41 wherein the liquid phase is substantially free of sulfuric acid.

44. The process according to claim 43, wherein the content of water in the liquid phase is lower than or equal to about 250 g/kg of liquid phase.

45. (canceled)

46. The process according to claim 1, carried out under continuous mode in a trickle-bed reactor.

47-49. (canceled)

50. A process for making a polyester comprising obtaining 1,3-propanediol according to the process of claim 1, and further submitting said 1,3-propanediol to a reaction with a carboxylic acid and/or a carboxylic acid ester.

51. A process for making a polyester fiber comprising obtaining a polyester according to the process of claim 50, and further converting said polyester in to a fiber.
